(19) United States
(12) Patent Application Publication
(10) Pub. No.: US 2016/0058585 A1
(43) Pub. Date: Mar. 3, 2016

(54) MEDICAL DEVICES INCLUDING COVERS AND METHODS FOR MAKING AND USING SAME

(71) Applicant: BOSTON SCIENTIFIC SCIMED, INC., Maple Grove, MN (US)

(72) Inventors: Dane T. Seddon, Boston, MA (US); Sean P. Fleury, Brighton, MA (US)

(21) Appl. No.: 14/838,051

(22) Filed: Aug. 27, 2015

Related U.S. Application Data
(60) Provisional application No. 62/043,826, filed on Aug. 29, 2014.

(51) Int. Cl. A61F 2/844

(52) U.S. Cl. CPC A61F 2/844; A61F 2210/0004

(57) ABSTRACT

An implantable medical device may include a stent being structured and arranged to increase in length from a deployed length to an extended length; and a cover 120 disposed around an exterior surface of a covered section of the stent, wherein a second portion of the cover overlaps a first portion of the cover, wherein an adhesive is disposed between the first portion of the cover and the second portion of the cover. Methods of using an implantable medical device and methods of making an implantable medical device are disclosed.

### CROSS-REFERENCE TO RELATED APPLICATIONS

This application claims the benefit of and priority to U.S. Provisional Application Ser. No. 62/043,826, filed Aug. 29, 2014, the entire contents of which are herein incorporated by reference.

### TECHNICAL FIELD

The present disclosure pertains to medical devices (e.g., stents) having covers. More particularly, the present disclosure pertains to medical devices (e.g., stents) having delayed, increased compliance.

### BACKGROUND

Medical devices, such as stents, have been used in a wide variety of medical applications for many years. For example, stents have been implanted in various body lumens, including a blood vessel, an esophagus, a trachea, a bronchus, lumens in the gastro-intestinal tract, and others.

In some applications, in order to avoid stent migration, stents are anchored in place over a period of time via ingrowth of biological tissue into one or more apertures or other structure in the sidewall of a medical device (e.g., stent). However, in some applications where ingrowth is not desired, covers (e.g., polymeric covers) have been applied to medical devices to inhibit such tissue ingrowth in the covered areas. Some medical devices have included covered sections in addition to uncovered sections.

Stents have been developed to allow for axial extension in response to a low amount of force to accommodate, in the case of an airway stent, instances of coughing, heavy breathing, swallowing, and sneezing. For example, a trachea or bronchus can increase in length up to 20% of its original length during the inhalation phase before promptly returning to its original length. However, stent designs generally have a limit of axial extension under low force before the stent is at a lock-out condition, which would require a much greater force for axial extension.

One problem that has arisen during stent deployment is that a stent may have the ability to settle into a body lumen (e.g., an airway, etc.) in one of a wide variety of configurations, including a compressed configuration (i.e., relatively shortened) and an extended configuration (i.e., relatively elongated or stretched). If a deployed stent settles in a body lumen (e.g., an airway) in the compressed configuration, tissue ingrowth may occur to anchor the stent in that position. In many cases, this delivery configuration allows the medical device to be sufficiently compliant to elongate with the airway with minimal resistance. If the stent settles in the expanded configuration, however, and tissue ingrowth occurs, the stent will already be at or near its maximum axial extension level. For a knitted stent, for example, the nature of the knitted design may lead the stent to lock out during breathing, coughing, etc. before the airway fully elongates, which would create increased resistance by the stent to the airway movement, which could result in several undesirable effects including a fractured stent, tearing of tissue at the locations of ingrowth, and/or inability for the patient to perform those movements properly or comfortably.

It is useful for medical devices (e.g., stents) to be positioned correctly to allow the device to anchor in a body lumen and have a desired level of compliance (e.g., axial compliance) while the stent is implanted. In some applications, such as implanting of an airway stent, a stent having an increased amount of axial compliance may be desired.

### SUMMARY

One aspect of the present disclosure is an implantable medical device including a stent having an exterior surface and a covered section extending from a first end section to a second end section, the stent being structured and arranged to increase in length from a deployed length to an extended length. The medical device also includes a cover disposed around the exterior surface of the covered section of the stent, wherein a second portion of the cover overlaps a first portion of the cover, wherein an adhesive is disposed between the first portion of the cover and the second portion of the cover.

Another aspect of the present disclosure is an implantable medical device having a delayed, increased compliance. The device includes a stent having an exterior surface and a covered section extending from a first end section to a second end section, the stent being structured and arranged to increase in length from a deployed length to an extended length. The device also includes a cover disposed around the exterior surface of the covered section of the stent, wherein the cover has an overall length extending from a first terminal end to a second terminal end, wherein the cover includes an overlap portion such that the distance from the first terminal end to the second terminal end is less than the overall length. The device further includes an adhesive disposed at the overlap portion, the adhesive being bioabsorbable and/or biodegradable. In one or more embodiments, the cover is structured and arranged to allow the device to exhibit a first compliance when the stent is deployed and to later exhibit a second, increased compliance when at least a portion of the adhesive is bioabsorbed or biodegraded.

Another aspect of the present disclosure is a method for deploying an implantable device in a lumen, the method including disposing an implantable medical device within a lumen, the implantable medical device comprising a cover disposed around an exterior surface of a covered section of a stent, wherein a second portion of the cover overlaps a first portion of the cover, wherein an adhesive is disposed between the first portion of the cover and the second portion of the cover. The method also includes allowing at least a portion of the adhesive to be degraded or absorbed, thereby increasing the compliance of the device.

Another aspect of the present disclosure is a method for making an implantable device. The method includes providing a stent having an exterior surface and disposing a cover around the exterior surface of the stent, wherein a second portion of the cover overlaps a first portion of the cover. The method also includes affixing the cover to the exterior surface of the stent at a first location proximal of the overlapping first portion and at a second location distal of the overlapping first portion and disposing a bioabsorbable and/or biodegradable adhesive between the first portion of the cover and the second portion of the cover.

In the methods and devices of the present disclosure, the adhesive may be bioabsorbable and/or biodegradable. For example, when a stent is implanted within a body lumen, at least some of the adhesive may biodegrade or bioabsorb after the stent has anchored to biological tissue that defines the body lumen. The adhesive may adhere to an exterior surface of at least one of the first portion and the second portion of the cover.

In the methods and devices of the present disclosure, the stent may be knitted. Increasing the length of the stent may cause the overlap portion of the cover to decrease in length. When the stent increases in length, the extended length may be at least 20 percent longer than the deployed length. Decreasing the length of the stent may cause the overlap portion of the cover to increase in length. Herein, the force required to lengthen the stent from the deployed length to an extended length may be reduced when the device exhibits the second compliance relative to the device exhibiting the first compliance.

Covers herein may include a third portion that overlaps the second portion, there being an annular fold between the second portion and third portion. The cover may include an annular fold between the first portion and the second portion. At least some of the first portion may slidingly engage at least some of the second portion when the stent changes length.

The cover may be structured and arranged to avoid ingrowth of biological tissue into the covered section when the stent is implanted in a body lumen. However, each of the first second sections of the stent may be uncovered and may be structured and arranged to allow ingrowth of biological tissue when implanted in a body lumen. The cover may be fixedly attached to the stent at two separated locations, wherein the length of the cover extending between the two locations exceeds the length of stent extending between the two locations. Covers herein may include two partially overlapping tubes and/or may be formed from a single piece of material.

The above summary of one or more aspects of the present disclosure is not intended to describe each disclosed embodiment or every implementation of the present disclosure. The detailed description, which follows, more particularly exemplifies these embodiments.

### BRIEF DESCRIPTION OF THE DRAWINGS

A detailed description is hereafter provided with specific reference being made to the drawings.

FIG. 1 shows a section view of a medical device in a contracted configuration according to one or more embodiments of the present disclosure;

FIG. 2 shows a section view of a medical device in an extended configuration according to one or more embodiments of the present disclosure;

FIG. 3 is a section view of a medical device having a discontinuous cover according to one or more embodiments of the present disclosure;

FIG. 4 is depiction of a medical device in a contracted configuration according to one or more embodiments of the present disclosure.

While the disclosure is amenable to various modifications and alternative forms, specifics thereof are shown by way of example in the drawings and are described in detail. It should be understood, however, that the intention is not to limit the present disclosure to the particular embodiments described. On the contrary, the intention is to cover all modifications, equivalents, and alternatives falling within the scope of the present disclosure.

### DETAILED DESCRIPTION

The subject matter of the present disclosure may alleviate or eliminate one or more of the problems mentioned above. The following description should be read with reference to the drawings, which are not necessarily to scale, wherein like reference numerals indicate like elements throughout the several views. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the claimed invention.

In at least one aspect of the present disclosure, an implantable medical device 100 is shown in FIG. 1. The device includes a stent 102 and a cover 120 having an adhesive 140 disposed between a first portion 130 of the cover 120 and a second portion 132 of the cover 120.

In one or more embodiments, the stent 102 has an exterior surface 104, an interior surface 106, and a covered section 112 that may extend from a first end section 110 to a second end section 114. In one or more embodiments, at least one of the first end section 110 and the second end section 114 is uncovered. Uncovered first end section 110 and uncovered second end section 114 may allow ingrowth of biological tissue such that medical device 100 to be anchored within a lumen (e.g., a body lumen) after implantation. In one or more embodiments, the ingrowth at the first and second end sections may be sufficient to avoid unintentional migration of the medical device along the body lumen (e.g., airway, etc.).

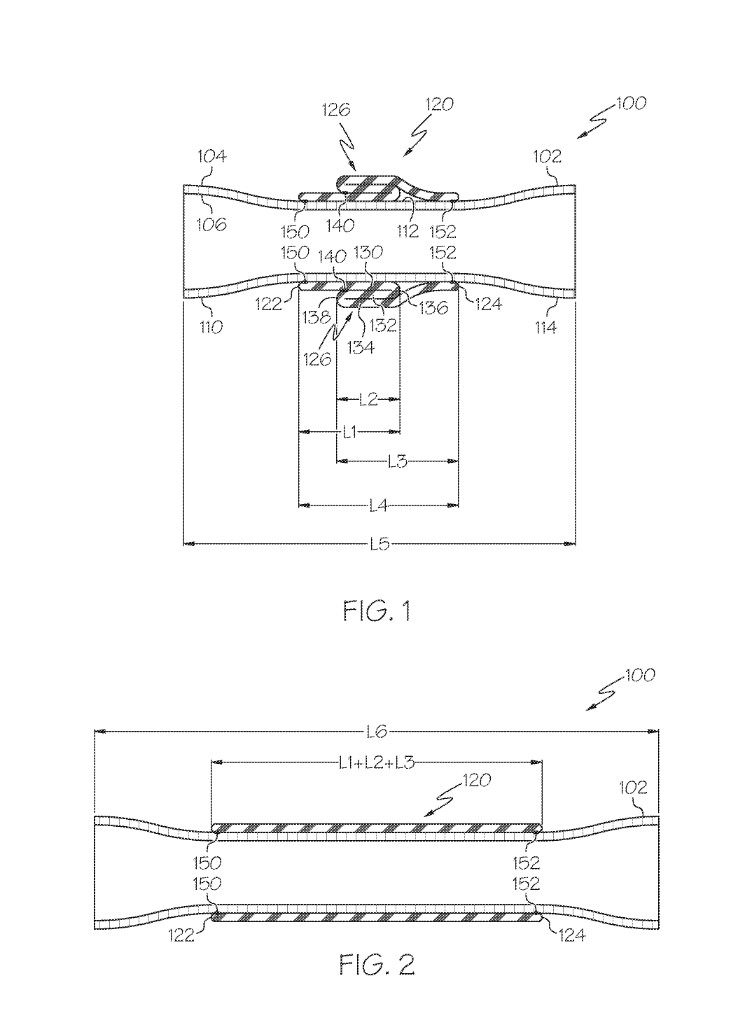

Stent 102 may be longitudinally extendible. For example, stent 102 may be structured and arranged to increase in length from a deployed length (L5 in FIG. 1) to an extended length (e.g., a length up to L6 in FIG. 2). In one or more embodiments, the extended length is greater than the length of the deployed length by at least 1% (e.g., by at least 5%, by at least 10%, by at least 15%, by at least 20%, etc.).

As shown in FIG. 1, a cover 120 is disposed around the exterior surface 104 of the covered section 112 of the stent 102. In one or more embodiments, the cover 120 is directly contacting the exterior surfaces of the stent 102, as shown in FIG. 1. However, in some embodiments, cover 120 may be disposed around the exterior surface 104 of the stent 102 with one or more layers or barriers between the cover 120 and the stent 102.

FIG. 1 shows that the second portion 132 of the cover 120 overlaps the first portion 130 of the cover 120. In one or more embodiments, as shown in FIG. 1, cover 120 may further include a third portion 134 that overlaps the second portion 132, there being an annular fold 138 (e.g., a circumferential fold, etc.) between (e.g., connecting, etc.) the second portion 132 and the third portion 134. In some embodiments, the cover includes an annular fold 136 (e.g., a circumferential fold) between (e.g., connecting, etc.) the first portion 130 and the second portion 132.

As can be seen in FIG. 1, cover 120 has an overall length (e.g., axial length) extending from a first terminal end 122 to a second terminal end 124, which includes extending from first terminal end 122 to annular fold 136 (e.g., L1 in FIG. 1), then to annular fold 138 (e.g., L2 in FIG. 1), and then to second terminal end 124 (e.g., L3 in FIG. 1). Thus, the overall length of cover 120 is L1+L2+L3. In one or more embodiments, the distance between the first terminal end 122 and the second terminal end 124 (see L4 in FIG. 1) is less than that overall length of the cover 120. In one or more embodiments, the overall length of cover 120 is at least 1% greater than (e.g., at least 10% greater than, at least 20% greater than, etc.) the distance between the first and second terminal ends 122, 124 of cover 120.

The device of FIG. 1 also includes an adhesive 140 disposed at the overlap portion 126 (e.g., between the first portion 130 of the cover 120 and the second portion 132 of the cover 120, between the second portion 132 and the third portion 134 of the cover 120, etc.). In one or more embodiments, the adhesive may extend longitudinally on a part of or all of first portion 130 and may extend outside of the overlap portion. Moreover, in some embodiments, the adhesive may extend partially or completely circumferentially around at least a portion of the first portion 130 of the cover 120.

In one or more embodiments, the adhesive is bioabsorbable and/or biodegradable. Any of a wide variety of bioabsorbable and/or biodegradable adhesives may be used, including, but not limited to, a starch glue, among other biodegradable and/or bioabsorbable adhesives. In one or more embodiments, the adhesive is a fibrin glue, a polyvinyl alcohol (PVA) glue, or a starch-based glue. In one or more embodiments, the adhesive may include one or more natural adhesives made from one or more organic sources and/or may include one or more synthetic adhesives.

In the present disclosure, the overlap portion has the effect of storing some additional distance in the overlap portion using folds and that is at least temporarily held in place using an adhesive (e.g., a bioabsorbable glue, a starch glue, a biodegradable glue, etc.).

After the stent has had time to anchor, the adhesive (e.g., glue, etc.) may deteriorate, allowing the cover (e.g., sleeve, etc.) to relax and reach an increased level of compliance. Disposing a bioabsorbable and/or biodegradable adhesive between the first and second portions of the cover provides a time-sensitive adhesiveness to the device 100 and a time-sensitive compliance to the medical device. For example, a particular adhesive may be selected such that the adhesive adheres the two portions of the cover for a time sufficient to allow ingrowth of biological tissue into one or more uncovered sections of the stent (e.g., the first and second end sections 110, 114). After the uncovered sections of the stent have anchored, then a sufficient amount of the adhesive may be bioabsorbed or biodegraded to reduce or eliminate the adhesiveness between the first and second portions of the cover, thereby providing a delayed, increased compliance (e.g., increased axial compliance, etc.) to the medical device.

In one or more embodiments, each of the first and second sections 110, 112 of the stent 102 is uncovered and is structured and arranged to allow ingrowth of biological tissue when implanted in a body lumen. Any of a wide variety of stent designs that allow for ingrowth of biological tissue may be utilized in the present disclosure. In one or more embodiments, cover 120 may be structured and arranged to avoid ingrowth of biological tissue into the covered section 112 when the stent 102 is implanted in a body lumen. Any of a wide variety of cover designs that inhibit ingrowth of biological tissue may be utilized in the present disclosure.

In one or more embodiments, the cover 120 is structured and arranged to allow the device to exhibit a first compliance (e.g., longitudinal compliance, axial compliance, etc.)

when the stent is deployed (e.g., at the time the stent is implanted) and to later exhibit a second, increased compliance when at least a portion of the adhesive is bioabsorbed or biodegraded. For example, a medical device of the present disclosure may be implanted with an adhesive adhering two portions of cover 120 together such that the medical device is implanted in an axially compressed configuration. While one or both stent ends are being anchored by tissue ingrowth, which may take hours, days, or weeks depending on the implantation location, the adhesive may be progressively bioabsorbed or biodegraded. Eventually, the extent of bioabsorption and biodegradation increases until at least a point in time where the adhesive cannot adhere the two portions of cover together. At that time, the medical device 100 may be substantially free to increase in length and/or contort to accommodate biological processes such as heavy breathing, sneezing, coughing, and swallowing.

In some embodiments, the cover is secured to the stent at two or more separate locations. For example, the cover may be secured to the stent at the proximal end (e.g., the proximal terminal end) of the cover and at the distal end (e.g., the distal terminal end) of the cover. In some embodiments, one or more portions of the cover may be secured to the stent at locations that are not at the distal or proximal end of the cover. The cover may be secured (e.g., affixed, bonded, adhered, attached, etc.) to the stent using any of a wide variety of chemical, mechanical, and/or electromagnetic securement structures including, but not limited to, a suture, an adhesive, a chemical bond, a solvent bond, a hook, a barb, or any other suitable securement structure. In one or more embodiments, the securement structure is intended to secure the cover to the stent throughout the useful life (e.g., permanently) of the medical device while implanted.

Between the two or more locations 150, 152 where the cover 120 is secured to the stent 102, the cover's length that extends between the two locations 150, 152 exceeds the length of the stent extending between the two locations 150, 152. As shown in FIG. 1, the cover's length that extends between the two locations 150, 152 includes the distances from each annual fold toward each of the two or more locations 150, 152 plus three times the length of the overlap, due to the overlap having three layers, whereas the length of the stent 102 between the two locations 150, 152 is merely the distance between the two locations 150, 152.

In one or more embodiments, cover 120 is formed from a single piece of material. As shown in FIG. 1, cover 120 is a tubular piece of material that folds back on itself to form an overlap portion, and then folds again back on the overlap portion. As can also be seen in FIG. 1, cover 120 may include two or more at least partially overlapping tubes, such as the tube including the first portion 130 (extending from second terminal end 124 to annular fold 136), the tube including the second portion 132 (extending between annular folds 136, 138), and the tube including the third portion 134 (extending from first terminal end 122 to annular fold 138).

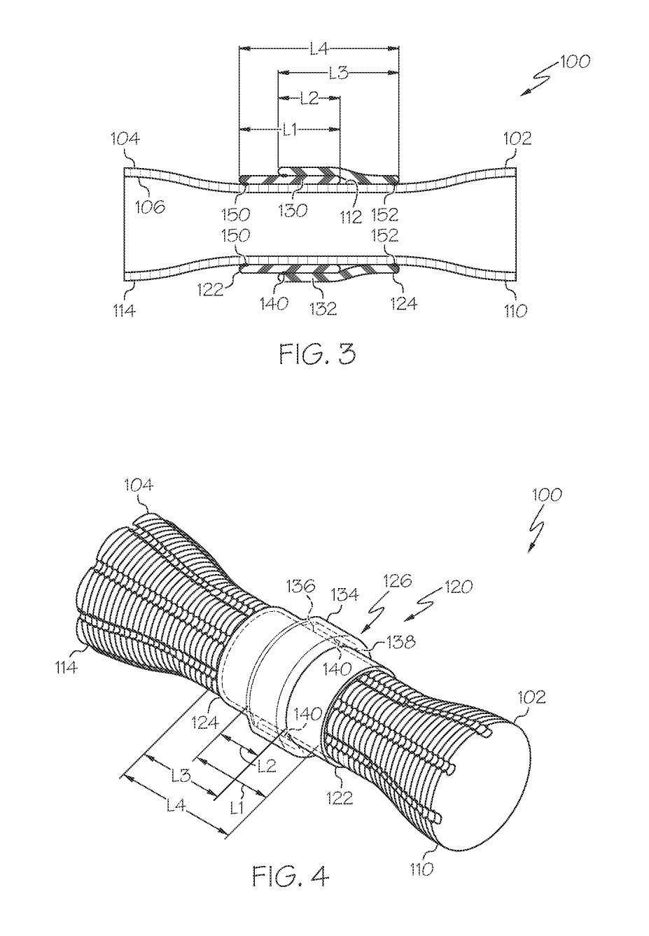

In some embodiments, the two or more at least partially overlapping tubes are not continuous (e.g., formed from two separate pieces of material), as shown in FIG. 3. In one or more embodiments of the present disclosure that include two separate covers that at least partially overlap, the length of the covers is taken to mean the sum of the lengths of each of the overlapping covers.

In the present disclosure, the adhesive 140 may be applied in any suitable location to adhere one portion of the cover to another portion of the cover. As shown in FIG. 1, adhesive 140 adheres an exterior surface of first portion 130 to an exterior surface (albeit inwardly facing) of second portion 132 of cover 120. In one or more embodiments, adhesive may be used to adhere two or more interior surfaces (e.g., an interior surface (albeit outwardly facing) of second portion 132 with an interior surface of third portion 134 of cover 120). In one or more embodiments, an adhesive may be used to adhere an interior surface of the cover to an exterior surface of the cover. For example, in FIG. 3, the exterior surface of first portion 130 is adhered to an interior surface of second portion 132 of cover 120.

A wide variety of stent designs are known. The methods and constructions described herein may be utilized with a stent having any suitable stent design. In one or more embodiments, the stent is of a knitted construction. In one or more embodiments, the stent may be knitted, woven, or braided, or a combination of these.

In one or more embodiments, increasing the length of the stent 102 causes the overlap portion 126 to decrease in length. That is, as the length of the stent is increased (due to, for example, a biological function), part of the second portion 132 may roll around one or both of the annular folds 136, 138 thereby increasing the length of the first and/or third portions and decreasing the length of the second portion. Thus, the overlap portion 126 of the cover 120 decreases in length in response to increases in stent length. As shown in FIG. 2, the cover may increase in length when the stent increases in length to an extended length (L6 in FIG. 2) until there is no more overlap portion and the distance between the first and second terminal ends is the same as the cover's overall length (L1+L2+L3). When the stent decreases in length to that shown in FIG. 1, the cover will also decrease in length by forming an overlap portion again.

Another aspect of the present disclosure relates to a method for deploying an implantable device as described herein in a lumen. For example, with reference to FIG. 1, the method includes disposing an implantable medical device 100 within a lumen. The implantable medical device 100 may include a cover 120 disposed around an exterior surface 104 of a covered section 112 of a stent 102, as shown in FIGS. 1 and 3. In one or more embodiments, a second portion 132 of the cover 120 overlaps a first portion 130 of the cover 120, wherein an adhesive 140 is disposed between the first portion 130 of the cover 120 and the second portion 132 of the cover 120.

The method includes allowing at least a portion of the adhesive 140 to be degraded or absorbed, thereby increasing the compliance of the device 100 as described herein. In one or more embodiments, allowing at least a portion of the adhesive to be degraded or absorbed includes exposing at least a portion of the adhesive to biological tissue and/or fluids and/or exposing at least a portion of the adhesive to one or more fluids (e.g., air, water, saliva, blood, etc.) that pass through the body lumen in which the medical device is implanted.

In one or more embodiments, the extent of overlap of the second portion of the cover over the first portion of the cover decreases when the stent increases in length. For example, when at least a portion of the adhesive biodegrades or is bioabsorbed, the first and second portions of the cover may then move relative to the other. In one or more embodiments, the first and second portions of the cover may include a sliding engagement wherein, for example, a second portion rolls around an annular fold and provides additional length for the first portion.

Another aspect of the present disclosure includes a method for making an implantable device of the present disclosure. The method includes providing a stent having an exterior surface, disposing a cover around the exterior surface of the stent, wherein a second portion of the cover overlaps a first portion of the cover, affixing the cover to the exterior surface of the stent at a first location proximal of the overlapping first portion and at a second location distal of the overlapping first portion, and disposing a bioabsorbable and/or biodegradable adhesive between the first portion of the cover and the second portion of the cover.

FIG. 4 provides a depiction of a medical device 100 including a stent 102 (e.g., a knitted stent) having a cover 120 disposed on an exterior surface of the stent 102. The stent 102 includes two ends that are uncovered by cover 120 and a covered section 112 extending between the two uncovered ends. The open structure of the uncovered ends provides an anti-migration feature to the medical device in that the open structure promotes tissue ingrowth in order to anchor the stent in place when the medical device is implanted in a body lumen. The stent of FIG. 4 represents, for example, an Ultraflex® stent available from Boston Scientific Corp. (Natick, Mass.), which is a knitted stent. The knitted design allows for axial extension even when relatively a low force is applied, up to a certain point where the knitted design locks out.

The axial extension is useful in, for example, airway stents where coughing, heavy breathing, swallowing, and sneezing, among other body functions, can cause the trachea and bronchi to extend up to 20% beyond its original length during, for example, the inhalation phase before returning to its original length. In one or more embodiments, little if any compression past the original length occurs because the cartilage rings bottom out on each other. In one or more embodiments, a stent that allows axial extension with very low forces is useful in order to reduce or eliminate the tendency of the stent to migrate within a body lumen.

In FIG. 4, the cover 120 includes an overlap portion 126 and is secured to the stent at a first location proximal of the overlap portion 126 and a second location distal of the overlap portion 126. The medical device of FIG. 4 also shows adhesive applied in two locations on a first portion of the cover 120 and between the first portion of the cover and a second portion of the cover 120. Adhesive 140 of FIG. 4 is shown to extend beyond the overlap portion.

When the stent of FIG. 4 is deployed within a body lumen, the adhesive adhering the two portions of cover together causes the stent to settle in the body lumen in a compressed configuration. This is useful because tissue ingrowth will occur to anchor the stent in that location and in that configuration. Then, having anchored in the body lumen in that configuration, allowing the adhesive to biodegrade or bioabsorb allows the medical device to be compliant and elongate with the airway with reduced resistance from the cover or from the stent locking out.

The medical devices and methods of the present disclosure allow a stent to be positioned correctly and with an appropriate level of compliance, not only for patient safety, but for patient comfort as well.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same or substantially the same function or result). In many instances, the term “about” may include numbers that are rounded to the nearest significant figure.

The recitation or disclosure of numerical ranges by endpoints includes all numbers within that range (e.g., 1 to 5 includes 1, 1.5, 2, 2.75, 3, 3.80, 4, and 5).

As used in this specification and the appended claims, the singular forms “a,” “an,” and “the” include plural references unless the context clearly dictates otherwise. As used in this specification and the appended claims, the term “or” is generally employed in its sense including “and/or” unless the context clearly dictates otherwise.

References in the specification to “an embodiment,” “some embodiments,” “one or more embodiments,” “other embodiments,” etc., indicate that the embodiment described may include a particular feature, structure, or characteristic, but not every embodiment necessarily includes the particular feature, structure, or characteristic. Moreover, such phrases do not necessarily refer to the same embodiment. Further, when a particular feature, structure, or characteristic is described in connection with one embodiment, it should be understood that such feature, structure, or characteristic may also be used in connection with other embodiments, whether or not explicitly described, unless clearly stated to the contrary.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one embodiment being used in other embodiments.

What is claimed is:

1. An implantable medical device comprising:
a stent having an exterior surface and a covered section extending from a first end section to a second end section, the stent being structured and arranged to increase in length from a deployed length to an extended length; and
a cover disposed around the exterior surface of the covered section of the stent, wherein a second portion of the cover overlaps a first portion of the cover, wherein an adhesive is disposed between the first portion of the cover and the second portion of the cover.

2. The device of claim 1 wherein the adhesive is bioabsorbable and/or biodegradable.

3. The device of claim 1 wherein the cover comprises an annular fold between the first portion and the second portion.

4. The device of claim 1 wherein the cover further comprises a third portion that overlaps the second portion, there being an annular fold between the second portion and the third portion.

5. The device of claim 1 wherein each of the first and second sections of the stent is uncovered and is structured and arranged to allow ingrowth of biological tissue when implanted in a body lumen.

6. The device of claim 1 wherein the cover is structured and arranged to avoid ingrowth of biological tissue into the covered section when the stent is implanted in a body lumen.

7. The device of claim 1 wherein the cover is fixedly attached to the stent at two separated locations, wherein the length of the cover extending between the two locations exceeds the length of stent extending between the two locations.

8. The device of claim 1 wherein at least some of the first portion slidingly engages at least some of the second portion when the stent changes length.

9. The device of claim 1 wherein the cover comprises two partially overlapping tubes.

10. The device of claim 1 wherein the cover is formed from a single piece of material.

11. The device of claim 1 wherein, when the stent is implanted within a body lumen, at least some of the adhesive biodegrades or bioabsorbs after the stent has anchored to biological tissue that defines the body lumen.

12. The device of claim 1 wherein the adhesive adheres to an exterior surface of at least one of the first portion and the second portion.

13. The device of claim 1 wherein the stent is knitted.

14. An implantable medical device having a delayed, increased compliance, the device comprising:
a stent having an exterior surface and a covered section extending from a first end section to a second end section, the stent being structured and arranged to increase in length from a deployed length to an extended length; and
a cover disposed around the exterior surface of the covered section of the stent, wherein the cover has an overall length extending from a first terminal end to a second terminal end, wherein the cover includes an overlap portion such that the distance from the first terminal end to the second terminal end is less than the overall length;
an adhesive disposed at the overlap portion, the adhesive being bioabsorbable and/or biodegradable;
wherein the cover is structured and arranged to allow the device to exhibit a first compliance when the stent is deployed and to later exhibit a second, increased compliance when at least a portion of the adhesive is bioabsorbed or biodegraded.

15. The device of claim 14 wherein the extended length is at least 20 percent longer than the deployed length.

16. The device of claim 14 wherein increasing the length of the stent causes the overlap portion to decrease in length.

17. The device of claim 15 wherein decreasing the length of the stent causes the overlap portion to increase in length.

18. The device of claim 14 wherein the force required to lengthen the stent from the deployed length to an extended length is reduced when the device exhibits the second compliance relative to the device exhibiting the first compliance.

19. A method for deploying an implantable device in a lumen, the method comprising:
disposing an implantable medical device within a lumen, the implantable medical device comprising a cover disposed around an exterior surface of a covered section of a stent, wherein a second portion of the cover overlaps a first portion of the cover, wherein an adhesive is disposed between the first portion of the cover and the second portion of the cover; and
allowing at least a portion of the adhesive to be degraded or absorbed, thereby increasing the compliance of the device.

20. The method of claim 19 wherein the extent of overlap of the first portion of the cover over the second portion of the cover decreases when the stent increases in length.
